(19) United States
(12) Patent Application Publication
(10) Pub. No.: US 2007/0162092 A1
(43) Pub. Date: Jul. 12, 2007

(54) LASER ACTIVATOR FOR PHYSICAL TREATMENT

(76) Inventor: Hsin-Chuan Yen, Yungho, Taipei County (TW)

Correspondence Address: PAI PATENT & TRADEMARK LAW FIRM, 1001 FOURTH AVENUE, SUITE 3200, SEATTLE, WA, 98154, US

(21) Appl. No.: 11/306,775

(22) Filed: Jan. 11, 2006

(51) Int. Cl. A61N 5/06

(52) U.S. Cl. 607/89

(57) ABSTRACT

A laser activator is disclosed to include a main unit, which has a power switch, a power indicator light, a power level adjustment switch, a power intensity indicator light, a laser light activation switch, a laser light working indicator light, a charging socket, a laser light connection jack, and a control circuit, a cable, and a laser contact unit, which is made in the form of an armpit piece, earpiece or vagina plug and electrically connected to the main unit through the cable for emitting low intensity laser to the armpit, ear or vagina to active the cells of the radiated body part.

### BACKGROUND OF THE INVENTION

(a) Field of the Invention

The present invention relates to a laser activator for physical treatment and more particularly to a laser activator which can be conveniently secured to the armpit, the external auditory canal of the ear or the vagina to radiate the contact area of the body with low intensity laser.

(b) Description of the Prior Art

In 1967, a few years after the first working laser was invented, Endre Mester in Semmelweis University, Budapest, Hungary took some mice, shaved the hair off their backs, divided them into two groups and gave a laser treatment with a low-power ruby laser to one group. The treated group did not get cancer and their hair grew back more quickly than the untreated group. That was how “laser biostimulation” was discovered. Professor Endre Mester started laser research in 1965 and first lectured on the use of the laser in medicine in 1966. In 1967, Endre Mester invented the Biostimulatory Effect of the Low Intensity Laser. He started to treat patients with non-healing skin ulcers in 1971 and his Low Intensity Laser Irradiation protocol became well known all over the world. Nowadays, low intensity laser has been intensively used in different applications, for example, laser knife in operation room. In an operation room, low intensity laser of about 50 W is used in cutting the body of the patient and stopping bleeding. For medical purposes, only skilled physicians are allowed to use low intensity laser. In recent years, laser power is also employed to remove speckles. However, except for the application to remove speckles, few safe laser apparatus have been disclosed for medical treatment.

The bodily constitution of a healthy person should be neutral to alkaline. With the increasing age and due to different habits in drinking and eating, a big amount of toxic substances, impurities, bacteria, germs, etc., are being accumulated in the body, thereby causing the bodily constitution to be biased from alkaline or neural status toward acidity, resulting in a big amount of positive charges in blood and a big amount of negative charges around the red blood cells and white blood cells. As a result, positive charges attract negative charges, thereby causing the red blood cells to be bonded together. The capillaries of human beings have a diameter about 5˜7 μm. When the blood becomes viscous, red blood cells cannot enter capillaries for circulation, and the organs at the ends of capillaries cannot obtain sufficient nutrition and oxygen or expel carbon dioxide, excessive water, toxic substances, impurities and germs to the outside of the body, thereby causing the capillary tissues and cells to decay, and the body of the person becomes unhealthy, or a tumor may develop in the body.

Biolaser uses low intensity laser to penetrate the subcutaneous tissues at about 5 cm. The application of low intensity laser is to directly radiate the auditory canal of the ear, the armpit and the vagina. There are capillaries in the auditory canal of the ear and the vagina, and artery and vein in the armpit at about 3 cm beneath the subcutaneous layer. Light interacts with blood cells in capillary to cause photosynthesis, causing red blood cells to be separated from white blood cells so that red blood cells can enter capillaries for circulation, enhancing metabolism and invigorating the weakened tissues and cells.

In case the capillaries or other delivery channels in the ears, armpits or vagina are blocked, the radiation of low intensity laser will activate the blood cells, thus improving the circulation of blood. Further, the ears, the armpits and the vagina are dark and wet, not easy to clean. These parts of the body tend to be infected with bacteria. Radiation of low intensity laser improves metabolism and blood circulation, thereby preventing infection.

### SUMMARY OF THE INVENTION

The present invention has been accomplished under the circumstances in view. It is one object of the present invention to provide a laser activator which uses a red laser light at a frequency of about 650 nm and power intensity of about 5 mw to radiate a part of the body of a person, thereby improving the metabolism and activating the growth of cells and separating red blood cells from white blood cells without damaging the tissues and cells of the body so as to improve the person's health. It is another object of the present invention to provide a laser activator which has a laser contact unit made in the form of an armpit piece, ear piece, or vagina plug for direct contact with the armpit, external auditory canal or vagina.

To achieve these and other objects of the present invention, the laser activator comprises a main unit, a laser contact unit, and a cable. The main unit comprises a power switch, a power indicator light, a power level adjustment switch, a power intensity indicator light, a laser light activation switch, a laser light working indicator light, a charging socket, a laser light connection jack, and a control circuit. The laser contact unit comprises at least one activating device. Each activating device comprises a shielding wire, a fiber optic pin coupling metal connector, a semiconductor laser emitter, a planoconvex lens, and a laser light output hole for output of laser light emitted by the semiconductor laser emitter. The cable has a first end electrically fixedly connected to the laser contact unit, and a second end provided with a connector that is connectable to the laser light connection jack of the main unit.

### BRIEF DESCRIPTION OF THE DRAWINGS

FIG. 1 is a perspective view of a laser activator in accordance with a first embodiment of the present invention.

FIG. 2 is a front view of the main unit of the laser activator according to the first embodiment of the present invention.

FIG. 3 is a schematic sectional view of the armpit piece of the laser activator according to the first embodiment of the present invention.

FIG. 4 is a schematic drawing showing an application example of the laser activator according to the first embodiment of the present invention.

FIG. 5 is a perspective view of a laser activator in accordance with a second embodiment of the present invention.

FIG. 6 is a schematic sectional view of one activating device of the laser activator according to the second embodiment of the present invention.

FIG. 7 is a schematic drawing showing one activating device of the laser activator inserted into the ear of a person according to the second embodiment of the present invention.

FIG. 8 is a schematic sectional view of one activating device of a laser activator in accordance with a third embodiment of the present invention.

FIG. 9 is a schematic drawing showing the use of a disposable sheath with the vagina plug type housing of the vagina plug type laser contact unit of the laser activator according to the third embodiment of the present invention.

FIG. 10 is a schematic drawing showing the vagina plug type housing of the vagina plug type laser contact unit of the laser activator of the third embodiment of the present invention inserted into the vagina of a female.

### DETAILED DESCRIPTION OF THE PREFERRED EMBODIMENTS

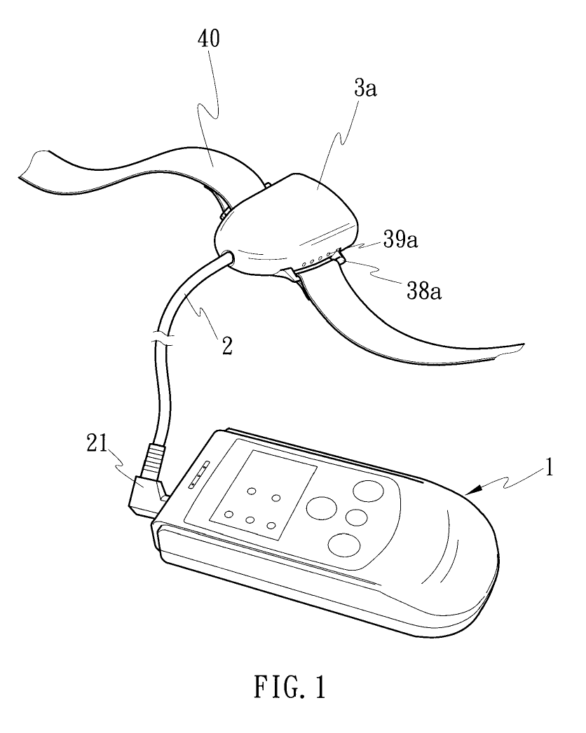

Referring to FIG. 1, a laser activator for physical treatment in accordance with one embodiment of the present invention is shown comprised of a main unit 1, a cable 2, and a laser contact unit (3a in FIG. 1).

The main unit 1 comprises on the outside a power switch 11 (that can be made to include an “on” button and an “off” button), a power indicator light 12, a power level adjustment switch 13 (for high/medium/low power intensity selection), a power intensity indicator light 14, a laser light activation switch 15 (the default duration can be set to be 30 minutes), a laser light working indicator light 16, a charging socket (not shown), a laser light connection jack 17, and on the inside a control circuit (not shown), a rechargeable battery (for example, a 3.6V, 1000 MA Li-battery for cellular telephone; not shown), and a battery charger (not shown; slow charging type, 110˜220VAC to DC5V, 500 MA) electrically coupled to the rechargeable battery.

Instead of the power indicator light 12, the power intensity indicator light 14, and the laser light working indicator light 16 described above, a display (not shown) can be used to indicate the status and level of the power and laser light.

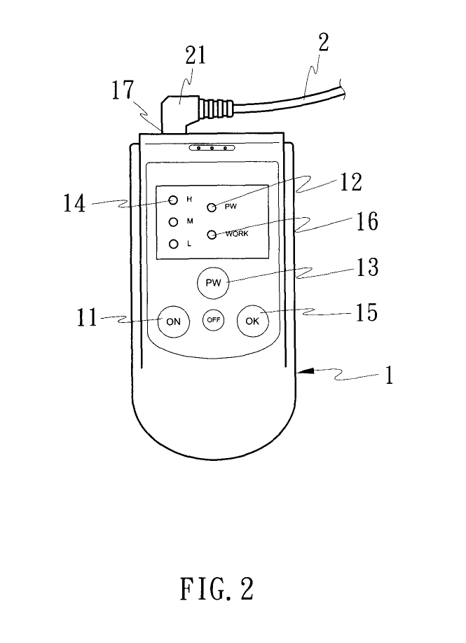

The cable 2, as shown in FIGS. 1 and 2, has one end provided with a plug 21 connectable to the laser light connection jack 17 of the main unit 1 and the other end directly connected to the laser contact unit.

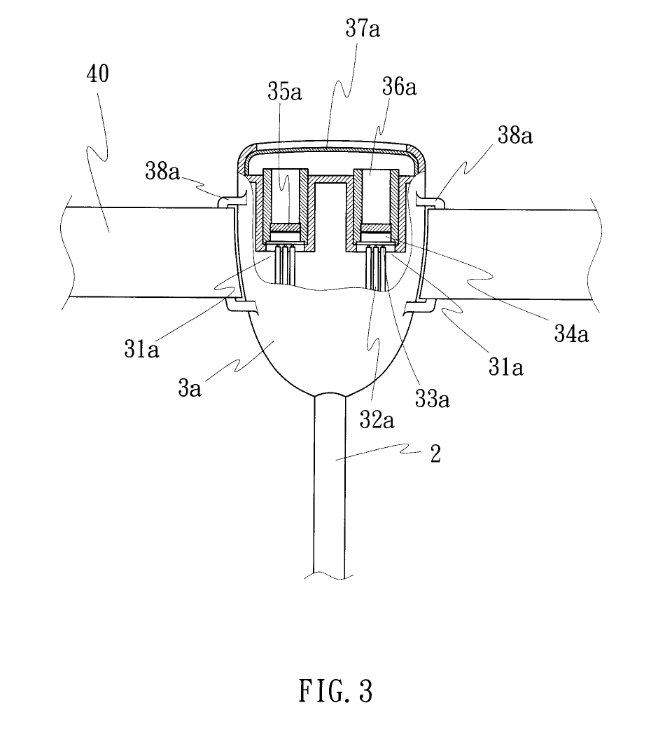

The aforesaid laser contact unit can be made in any of a variety of forms. FIG. 3 shows one embodiment of the laser contact unit. According to this embodiment, the laser contact unit is an armpit piece type laser contact unit 3a. The armpit piece type laser contact unit 3a comprises two activating devices 31a arranged in parallel, a transparent filter lens 37a that protects the activating devices 31a against armpit sweat and guides the laser light from the activating devices 31a forwards. Each activating device 31a comprises a shielding wire 32a, a metal connector for fiber optic pin coupling 33a, a semiconductor laser emitter 34a, a planoconvex lens 35a, and a laser light output hole 36a. The semiconductor laser emitter 34a is adapted to emit visible red laser light at 650 nm and 5 mw.

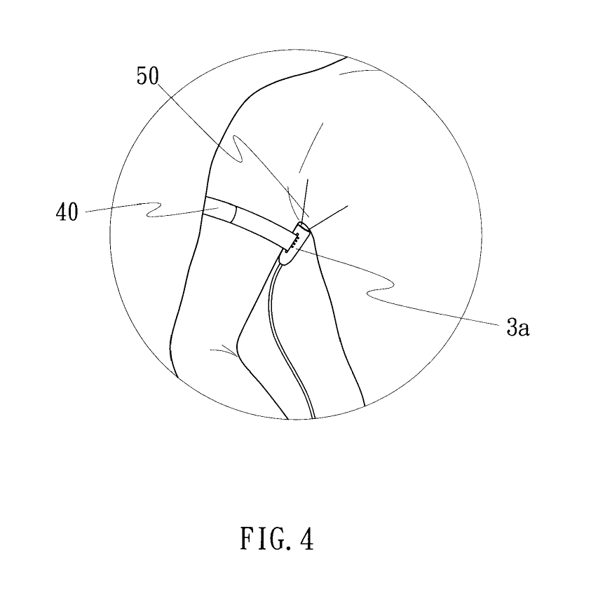

The armpit piece type laser contact unit 3a further has two lugs 38a disposed at two sides, a plurality of air vents 39a respectively disposed near the lugs 38a for dissipation of heat, and a fastening strap 40 inserted through the lugs 38a for securing the armpit piece type laser contact unit 3a to the user's arm, keeping the transparent filter lens 37a of the armpit piece type laser contact unit 3a in close contact with the armpit 50 (see FIG. 4). The fastening strap 40 is detachable, so that each individual user can use a respective fastening strap. Further, the fastening strap 40 can be in the form of an elastic band. Alternatively, the fastening strap 40 can be formed of a belt having the ends detachably joined by means of hook and loop materials, Velcros, or tie ropes.

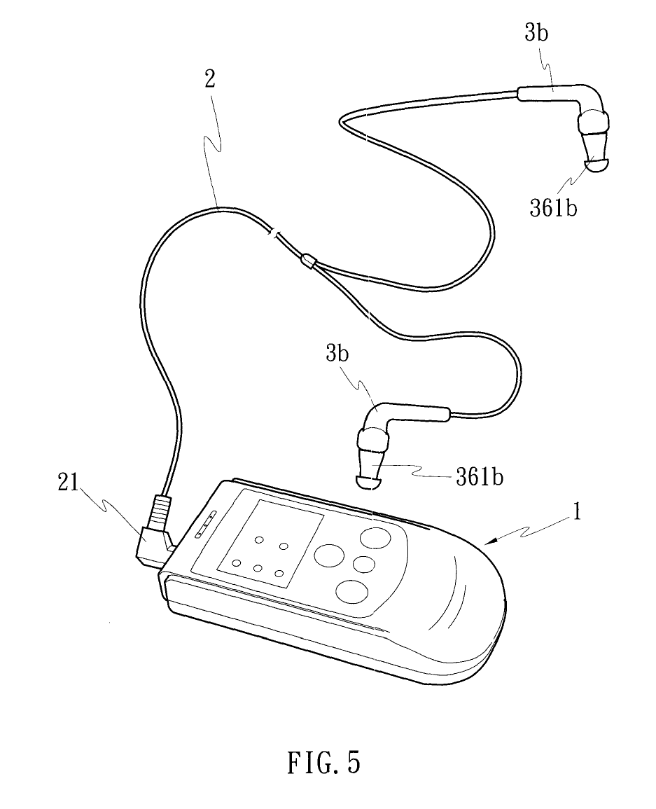

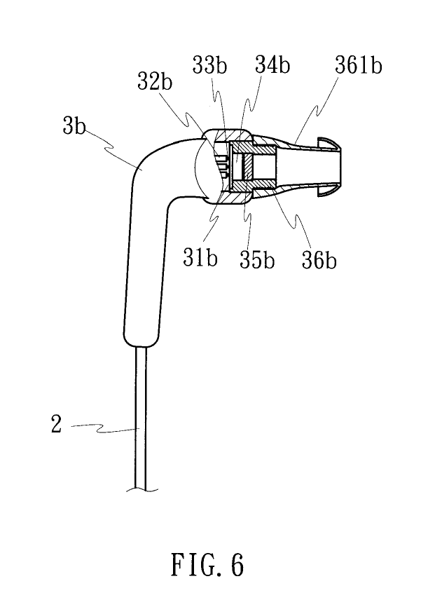

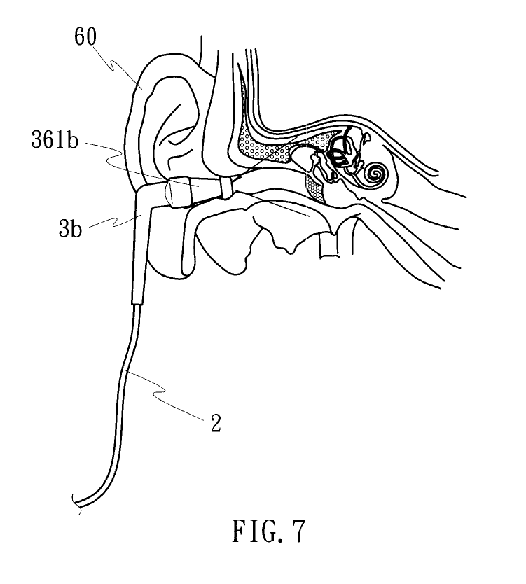

FIGS. 5˜7 show another embodiment of the laser contact unit. According to this embodiment, the laser contact unit is an earpiece type laser contact unit 3b. According to this embodiment, the earpiece type laser contact unit 3b comprises two activating devices 31b separately connected to one end of the cable 2. Each activating device 31b comprises a shielding wire 32b, a metal connector for fiber optic pin coupling 33b, a semiconductor laser emitter 34b, a planoconvex lens 35b, and a laser light output hole 36b. Each activating device 31b further comprises an earplug type housing 361b insertable into one ear 60 of the user (see FIG. 7). The earplug type housing 361b has a soft covering for positioning in the ear 60 securely and comfortably.

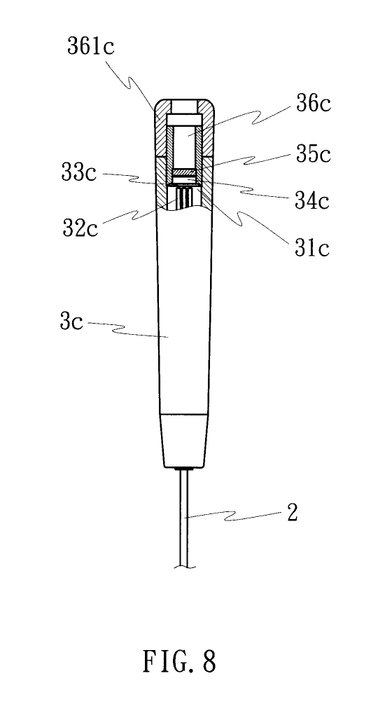

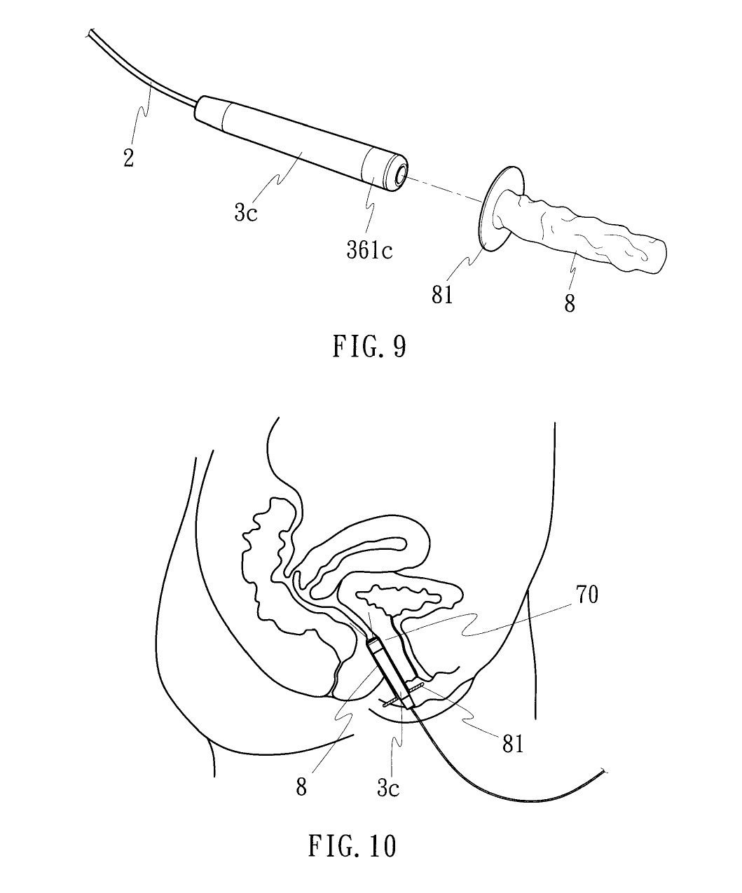

FIG. 8 shows still another embodiment of the laser contact unit. According to this embodiment, the laser contact unit is a vagina plug type laser contact unit 3c. According to this embodiment, the vagina plug type laser contact unit 3c comprises an activating device 31c connected to one end of the cable 2. The activating device 31c comprises a shielding wire 32c, a metal connector for fiber optic pin coupling 33c, a semiconductor laser emitter 34c, a planoconvex lens 35c, and a laser light output hole 36c. The activating device 31c further comprises a vagina plug type housing 361c insertable into the user's vagina 70 (see FIG. 10). A disposable sheath 8 may be placed over the vagina plug type housing 361c of the vagina plug type laser contact unit 3c and inserted with the vagina plug type housing 361c into the user's vagina 70 to prevent contamination (see FIG. 9). The sheath 8 has a stop flange 81 that is stopped outside the user's vagina 70 after insertion of the sheath 8 with the vagina plug type housing 361c into the user's vagina 70.

The bodily constitution of a healthy person should be neutral to alkaline. With the increasing age and due to different habits in drinking and eating, a big amount of toxic substances, impurities, bacteria, germs, etc., are being accumulated in the body, thereby causing the bodily constitution to be biased from alkaline or neural status toward acidity, resulting in a big amount of positive charges in blood and a big amount of negative charges around the red blood cells and white blood cells. As a result, positive charges attract negative charges, thereby causing the red blood cells to be bonded together. The capillaries of human beings have a diameter about 5˜7 μm. When the blood becomes viscous, red blood cells cannot enter capillaries for circulation, and the organs at the ends of capillaries cannot obtain sufficient nutrition and oxygen or expel carbon dioxide, excessive water, toxic substances, impurities and germs to the outside of the body, thereby causing the capillary tissues and cells to decay, and the body of the person becomes unhealthy. The invention is adapted to radiate the capillaries in the armpits, ears or vaginas with low intensity laser, causing photosynthesis in red blood cells, and therefore red blood cells are separated from white blood cells and become active. When red blood cells become active, they can enter capillaries for circulation, enhancing metabolism and invigorating the weakened tissues and cells.

When the laser activator is in use, the eyes should be kept away from direct light from the laser activator. Press the laser light activation switch 15 only after the armpit piece type laser contact unit 3a, earpiece type laser contact unit 3b or vagina plug type laser contact unit 3c has been secured to the armpit, ear or vagina. When the blood circulation becomes unsmooth, the infected area may lack oxygen, the reaction may become clumsy, and the menstruation may become unstable, thereby causing stomachache, headache, or fainting. The use of the present invention activates the cells and enhances the circulation of blood, thereby improving the physical conditions of the body. Healthy cells effectively prevent inception and accumulation of virus. Frequent use of the present invention to radiate different parts of the body with low intensity laser greatly improves the physical conditions of the body, making the body active and healthy.

A prototype of laser activator has been constructed with the features of FIGS. 1˜10. The laser activator functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A laser activator comprising:
a main unit, said main unit comprising a power switch, a power indicator light, a power level adjustment switch, a power intensity indicator light, a laser light activation switch, a laser light working indicator light, a charging socket, a laser light connection jack, and a control circuit;
a laser contact unit, said laser contact unit comprising at least one activating device, said at least one activating device each comprising a shielding wire, a fiber optic pin coupling metal connector, a semiconductor laser emitter, a planoconvex lens, a laser light output hole for output of laser light emitted by said semiconductor laser emitter; and
a cable, said cable having a first end electrically fixedly connected to said laser contact unit and a second end provided with a connector connectable to said laser light connection jack of said main unit.

2. The laser activator as claimed in claim 1, wherein said laser contact unit is an armpit piece type laser contact unit comprising two activating devices fixedly arranged in parallel on the inside thereof, a transparent filter lens adapted to protect the two activating devices against armpit sweat and to guide laser light from the semiconductor laser emitter of each of the two activating devices toward a predetermined direction, two lugs disposed at two sides of the two activating devices, a plurality of air vents respectively disposed near said lugs, and a fastening strap inserted through said lugs for securing the armpit piece type laser contact unit to the user's arm.

3. The laser activator as claimed in claim 1, wherein said laser contact unit is an earpiece type laser contact unit comprising two activating devices separately connected to the first end of said cable, the two activating devices each having an earplug type housing insertable into one ear of a person.

4. The laser activator as claimed in claim 1, wherein said laser contact unit is a vagina plug type laser contact unit comprising an activating device connected to the first end of said cable, the activating device having a vagina plug type housing insertable into the vagina of a female.

5. The laser activator as claimed in claim 4, further comprising a disposable sheath to be placed over said vagina plug type housing and inserted with said vagina plug type housing into the vagina of a female to prevent contamination.

6. The laser activator as claimed in claim 1, wherein said semiconductor laser emitter is adapted to emit visible red laser light at 650 nm and 5 mw.

7. A laser activator comprising:
a main unit, said main unit comprising a power switch, a power level adjustment switch, a laser light activation switch, a power and laser light status display, a charging socket, a laser light connection jack, and a control circuit;
a laser contact unit, said laser contact unit comprising at least one activating device, said at least one activating device each comprising a shielding wire, a fiber optic pin coupling metal connector, a semiconductor laser emitter, a planoconvex lens, a laser light output hole for output of laser light emitted by said semiconductor laser emitter; and
a cable, said cable having a first end electrically fixedly connected to said laser contact unit and a second end provided with a connector connectable to said laser light connection jack of said main unit.
